(19) United States
(12) Patent Application Publication
(10) Pub. No.: US 2017/0348029 A1
(43) Pub. Date: Dec. 7, 2017

(54) RCDF Instrument, Apparatus and Procedures

(71) Applicant: SpineCraft, LLC, Westmont, IL (US)

(72) Inventor: Wagdy Asaad, Burr Ridge, IL (US)

(21) Appl. No.: 15/170,865

(22) Filed: Jun. 1, 2016

(51) Int. Cl. A61B 17/70; A61B 17/56; A61B 17/68

(52) U.S. Cl. CPC A61B 17/7076; A61B 17/7032; A61B 2017/681; A61B 2017/564

(57) ABSTRACT

Instruments, apparatus and procedures for performing surgical operations are provided. A sleeve for performing surgical procedures may include an elongate main body; a channel extending within a length of the elongate main body; and a retainer configured and dimensioned to releasably retain a fixation member in the sleeve at a predetermined orientation and predetermined distance from a distal end of the sleeve. An assembly for performing surgical procedures may include a sleeve comprising: an elongate main body, a channel extending within a length of the elongate main body; a fixation member having been preloaded in the sleeve; and a bone fastener comprising: a shaft configured to engage bone, a tulip configured to receive a rod, a retention feature and extended tabs extending from the tulip.

### FIELD OF THE INVENTION

The present invention relates to the field of orthopedic surgery, in particular to devices, systems and methods for stabilizing and/or fixing bones and/or joints in a patient. More particularly, the present invention relates to instruments, assemblies and methods for surgical procedures on the spine.

### BACKGROUND OF THE INVENTION

Bone may be subject to degeneration caused by trauma, disease, and/or aging. Degeneration may destabilize bone and affect surrounding structures. For example, destabilization of a spine may result in alteration of a natural spacing between adjacent vertebrae. Alteration of a natural spacing between adjacent vertebrae may subject nerves that pass between vertebral bodies to pressure. Pressure applied to the nerves may cause pain and/or nerve damage. Maintaining the natural spacing between vertebrae may reduce pressure applied to nerves that pass between vertebral bodies. A spinal stabilization procedure may be used to maintain the natural spacing between vertebrae and promote spinal stability.

The fixation and/or stabilization of bones and/or bone fragments is/are commonly required by orthopedic surgeons to treat injuries such as fractures or disease. To accomplish this, the bones/bone fragments can be joined by a rod, plate or the like, which is fixed to the bones/bone fragments via fasteners such as screws, pins or the like. The connection by the rod(s), plate(s) or the like maintains the bones/bone fragments in a desired orientation and/or at desired spacings, positions, etc.

In spinal surgery, it is often necessary to secure various implants to the vertebrae and interconnect the vertebrae by attaching one or more rods or plates to the implants. Procedures such as reduction, compression and/or distraction are commonly carried out in an effort to repair fractures or disease caused by trauma, disease, genetic abnormalities and/or aging.

Conventional stabilization systems may require a large incision and/or multiple incisions in the soft tissue to provide access to a portion of the spine to be stabilized. Conventional procedures may result in trauma to the soft tissue, for example, due to muscle stripping. Minimally invasive techniques are being developed to minimize the size of incisions/degree of opening of the patient required to perform surgical procedures on the spine.

There is a continuing need for instruments, assemblies and procedures to facilitate such minimally invasive procedures.

### SUMMARY OF THE INVENTION

According to one aspect of the present invention, a sleeve for performing surgical procedures includes: an elongate main body; a channel extending within a length of the elongate main body; and a retainer configured and dimensioned to releasably retain a fixation member in the sleeve at a predetermined orientation and predetermined distance from a distal end of the sleeve.

According to at least one embodiment, the retainer is resiliently deformable to receive the fixation member.

According to at least one embodiment, the retainer comprises at least one leaf spring.

According to at least one embodiment, the retainer comprises a pair of opposing leaf springs.

According to at least one embodiment, the retainer comprises at least one prong to releasably capture the fixation member.

According to at least one embodiment, a distal end portion of the sleeve is configured and dimensioned to attach to a tulip portion of a bone fastener.

According to at least one embodiment, the sleeve further comprises a tool interface configured to mate with a working end of a counter-torque tool.

According to at least one embodiment, the sleeve further includes a passage formed through opposite walls of a distal end portion of the sleeve, the passage being configured to allow a rod or other connector to pass therethrough.

According to at least one embodiment, the sleeve further includes slots in the elongate main body configured and dimensioned to receive extended tabs that extend from a bone fastener.

According to at least one embodiment, the slots comprise stops that are configured to mate with sides of the extended tabs to prevent rotation of the sleeve relative to the extended tabs.

According to at least one embodiment, the sleeve further includes an alignment feature provided on an inner surface of a wall of the elongate main body, configured to properly align an orientation of the sleeve in a height direction relative to a bone fastener, when the sleeve is attached to the bone fastener.

According to at least one embodiment, the alignment feature comprises a ledge or shoulder.

According to at least one embodiment, the sleeve further includes stays forming anti-splaying slots with the a wall of the elongate main body, wherein the anti-splaying slots are configured to receive proximal end portions of the extended tabs and the stays are configured to prevent splaying of the extended tabs.

According to at least one embodiment, the sleeve further includes the fixation member being releasably retained by the retainer at the predetermined orientation and predetermined distance from the distal end of the sleeve.

According to at least one embodiment, the fixation member comprises a set screw.

According to another aspect of the present invention a sleeve for performing surgical procedures includes: an elongate main body; a channel extending within a length of the elongate main body; slots in the elongate main body configured and dimensioned to receive extended tabs that extend from a bone fastener; and an alignment feature provided on an inner surface of a wall of the elongate main body, configured to properly align an orientation of the sleeve in a height direction relative to a bone fastener, when the sleeve is attached to the bone fastener.

According to at least one embodiment, the slots comprise stops that are configured to mate with sides of the extended tabs to prevent rotation of the sleeve relative to the extended tabs.

According to at least one embodiment, the alignment feature comprises a ledge or shoulder.

According to at least one embodiment, the sleeve further includes stays forming anti-splaying slots with the a wall of the elongate main body, wherein the anti-splaying slots are configured to receive proximal end portions of the extended tabs and the stays are configured to prevent splaying of the extended tabs.

According to another aspect of the present invention, an assembly for performing surgical procedures includes: a sleeve comprising: an elongate main body; and a channel extending within a length of the elongate main body; a fixation member having been preloaded in the sleeve; and a bone fastener comprising: a shaft configured to engage bone; a tulip configured to receive a rod; a retention feature; and extended tabs extending from the tulip; wherein a distal end portion of the sleeve is mounted over the tulip; wherein the extended tabs are received in slots in the sleeve; and wherein the fixation member is located at a predetermined orientation and distance from a distal end of the sleeve to approximate a proximal end of the retention feature.

According to at least one embodiment, the sleeve further includes a retainer that releasably retains the fixation member in the sleeve at the predetermined orientation and the predetermined distance from the distal end of the sleeve.

According to at least one embodiment, the retainer is resiliently deformable to receive the fixation member.

According to at least one embodiment, the sleeve further includes a tool interface configured to mate with a working end of a counter-torque tool.

According to at least one embodiment, the assembly further includes a counter-torque tool, the working end of the counter-torque tool engaged with the tool interface.

According to at least one embodiment, the sleeve further includes a passage formed through opposite walls of the distal end portion of the sleeve, the passage being configured to allow the rod or other connector to pass therethrough.

According to at least one embodiment, the assembly further includes slots in the elongate main body, wherein the extended tabs are received in the slots.

According to at least one embodiment, the assembly further includes stops that mate with sides of the extended tabs to prevent rotation of the sleeve relative to the extended tabs.

According to at least one embodiment, the sleeve further includes an alignment feature provided on an inner surface of a wall of the elongate main body, configured to properly align an orientation of the sleeve in a height direction relative to the bone fastener.

According to at least one embodiment, the sleeve further includes stays forming anti-splaying slots with a wall of the elongate main body, wherein the anti-splaying slots receive proximal end portions of the extended tabs and the stays prevent splaying of the extended tabs.

According to at least one embodiment, the fixation member comprises a set screw.

According to another aspect of the present invention, a method of performing a surgical procedure includes: providing an elongate sleeve having proximal and distal ends and a channel extending therethrough; releasably retaining a fixation member in the channel of the sleeve at a predetermined orientation and predetermined distance from the distal end of the sleeve; and assembling the sleeve on a bone fastener.

According to at least one embodiment, the fixation member is preloaded into the channel prior to the assembling.

According to at least one embodiment, the fixation member is resiliently and releasably retained by a retainer.

According to at least one embodiment, the retainer is spring loaded.

According to at least one embodiment, the fixation member comprises a set screw.

According to at least one embodiment, the fixation member is preloaded by:

mounting the fixation member on a loading block; and advancing the fixation member and the loading block into the channel from the distal end of the sleeve.

According to at least one embodiment, the fixation member is advanced against a retainer that resiliently deforms to allow the fixation member to pass by and resiliently returns to an unbiased configuration to releasably retain the fixation member.

According to at least one embodiment, the method further includes: mating the fixation member with a retention member of the bone fastener.

According to at least one embodiment, the method further includes performing an operation on the bone fastener by manipulating the sleeve.

According to at least one embodiment, the operation includes at least one of reduction, distraction or compression.

According to at least one embodiment, the method further includes finally fixing the fixation member in the tulip.

According to at least one embodiment, the method further includes removing the sleeve from the bone fastener and breaking off the extended tabs.

These and other features of the invention will become apparent to those persons skilled in the art upon reading the details of the invention as more fully described below.

### BRIEF DESCRIPTION OF THE DRAWINGS

In the course of the detailed description to follow, reference will be made to the attached drawings. These drawings show different aspects of the present invention an, where appropriate, reference numerals illustrating like structures, components, materials and/or elements in different figures are labeled similarly. It is understood that various combinations of the structures, components, materials and/or elements, other than those specifically shown, are contemplated and are within the scope of the present invention.

FIG. 1 is a perspective view of a sleeve according to an embodiment of the present invention.

FIG. 2 shows an assembly in which a sleeve has been attached to a bone fastener in an operational state, in accordance with an embodiment of the present invention.

FIG. 3 shows the assembly of FIG. 2 having been rotated about its longitudinal axis by ninety degrees.

FIG. 4 is a cross-sectional view of FIG. 2 taken along line 4-4 in FIG. 2.

FIG. 5 is an enlarged, partially see-through, partial view of the assembly taken from box 5 in FIG. 4.

FIG. 6 illustrates an assembly process in progress according to an embodiment of the present invention.

FIG. 7 illustrates the assembly with an indication of direction of force for disassembling.

FIG. 8 is an enlarged, partial view of the portion of FIG. 7 contained within box 8.

FIG. 9 shows a longitudinal sectional view of the portion shown in FIG. 8.

FIG. 10 is a detailed, partially transparent view of the portion of assembly contained within circle 10-10 in FIG. 2.

FIGS. 11A-11E illustrate components and procedures for pre-loading a fixation member in accordance with an embodiment of the present invention.

FIG. 11F is a longitudinal sectional view of fixation member aligned and oriented with respect to a retention feature, according to an embodiment of the present invention.

FIG. 12 shows a counter-torque tool engaged with a sleeve of an assembly according to an embodiment of the present invention.

### DETAILED DESCRIPTION OF THE INVENTION

Before the present instruments, apparatus and procedures are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a tab” includes a plurality of such tabs and reference to “the rod” includes reference to one or more rods and equivalents thereof known to those skilled in the art, and so forth.

The publications discussed herein are provided solely for their disclosure prior to the filing date of the present application. The dates of publication provided may be different from the actual publication dates which may need to be independently confirmed.

As used herein “RCDF” refers to retraction, compression, distraction and/or final fixation.

The present invention is provided for minimally invasive procedures on the spine, but may also be used in conventional open procedures, or in orthopedic procedures involving bones other that the bone of the spine.

It is known to use minimally invasive procedures for limiting an amount of trauma to soft tissue surrounding vertebrae to be stabilized, see, for example, US Patent Application Publication No. 2008/0077139 A1, which is hereby incorporated herein, in its entirety, by reference thereto.

A spinal stabilization system may be used to achieve rigid pedicle fixation while minimizing the amount of damage to surrounding tissue. In some embodiments, a spinal stabilization system may be used to provide stability to two adjacent vertebrae (i.e., one vertebral level). A spinal stabilization system may include two bone fastener assemblies. One bone fastener assembly may be positioned in each of the vertebrae to be stabilized. An elongated member may be coupled and secured to the bone fastener assemblies. As used herein, “coupled” components may directly contact each other or may be separated by one or more intervening members. In some embodiments, a single spinal stabilization system may be installed in a patient. Such a system may be referred to as a unilateral, single-level stabilization system or a single-level, two-point stabilization system. In some embodiments, two spinal stabilization systems may be installed in a patient on opposite sides of a spine. Such a system may be referred to as a bilateral, single-level stabilization system or a single-level, four-point stabilization system.

In some embodiments, a spinal stabilization system may provide stability to three or more vertebrae (i.e., two or more vertebral levels). In a two vertebral level spinal stabilization system, the spinal stabilization system may include three bone fastener assemblies. One bone fastener assembly may be positioned in each of the vertebrae to be stabilized. An elongated member may be coupled and secured to the three bone fastener assemblies. In some embodiments, a single two-level spinal stabilization system may be installed in a patient. Such a system may be referred to as a unilateral, two-level stabilization system or a two-level, three-point stabilization system. In some embodiments, two three-point spinal stabilization systems may be installed in a patient on opposite sides of a spine. Such a system may be referred to as a bilateral, two-level stabilization system or a two-level, six-point stabilization system.

In some embodiments, combination systems may be installed. For example, a two-point stabilization system may be installed on one side of a spine, and a three-point stabilization system may be installed on the opposite side of the spine. The composite system may be referred to a five-point stabilization system.

Minimally invasive procedures may reduce trauma to soft tissue surrounding vertebrae that are to be stabilized. Only a small opening may need to be made in a patient. For example, for a single-level stabilization procedure on one side of the spine, the surgical procedure may be performed through a 2 cm to 4 cm incision formed in the skin of the patient. In some embodiments, the incision may be above and substantially between the vertebrae to be stabilized. In some embodiments, the incision may be above and between the vertebrae to be stabilized. In some embodiments, the incision may be above and substantially halfway between the vertebrae to be stabilized. Dilators, a targeting needle, and/or a tissue wedge may be used to provide access to the vertebrae to be stabilized without the need to form an incision with a scalpel through muscle and other tissue between the vertebrae to be stabilized. A minimally invasive procedure may reduce an amount of post-operative pain felt by a patient as compared to invasive spinal stabilization procedures. A minimally invasive procedure may reduce recovery time for the patient as compared to invasive spinal procedures.

Components of spinal stabilization systems may be made of materials including, but not limited to, titanium, titanium alloys, stainless steel, ceramics, and/or polymers. Some components of a spinal stabilization system may be autoclaved and/or chemically sterilized. Components that may not be autoclaved and/or chemically sterilized may be made of sterile materials. Components made of sterile materials may be placed in working relation to other sterile components during assembly of a spinal stabilization system.

Spinal stabilization systems may be used to correct problems in lumbar, thoracic, and/or cervical portions of a spine. Various embodiments of a spinal stabilization system may be used from the C1 vertebra to the sacrum. For example, a spinal stabilization system may be implanted posterior to the spine to maintain distraction between adjacent vertebral bodies in a lumbar portion of the spine.

Upon establishing the implantation of bone fastener assemblies to the vertebrae to be stabilized, various procedures are performed to place the vertebrae in the desired positions and orientations to be held under stabilization. Such procedures may include, but are not limited to reduction, compression and distraction. The present invention facilitates such procedures thus improve the ease of performing and success rates of minimally invasive procedure.

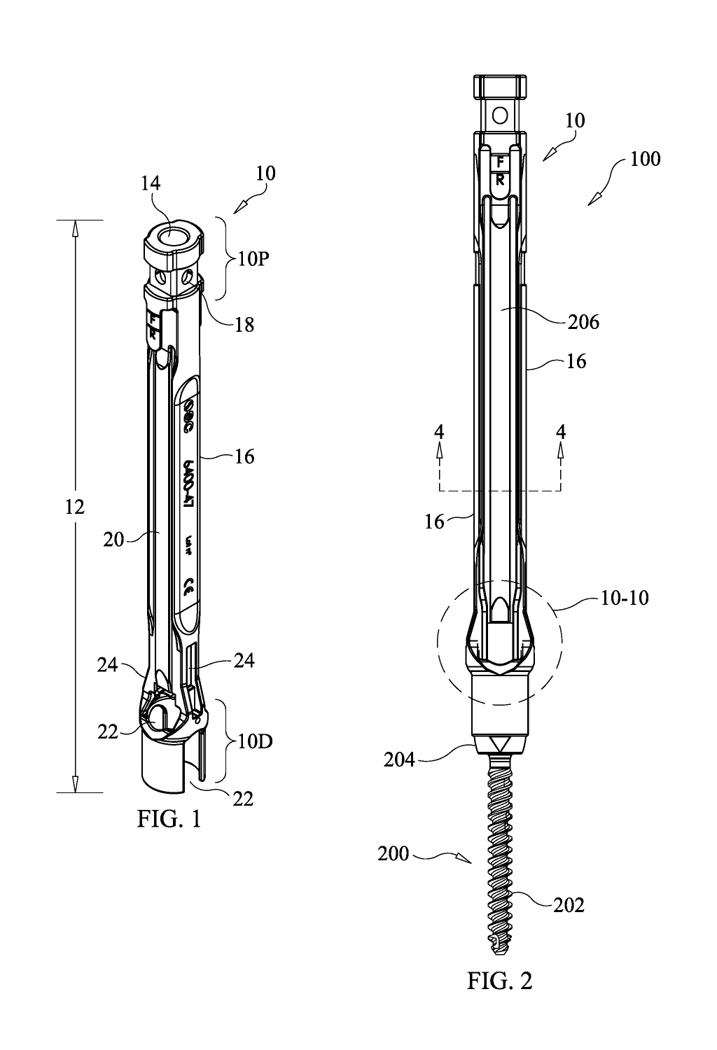

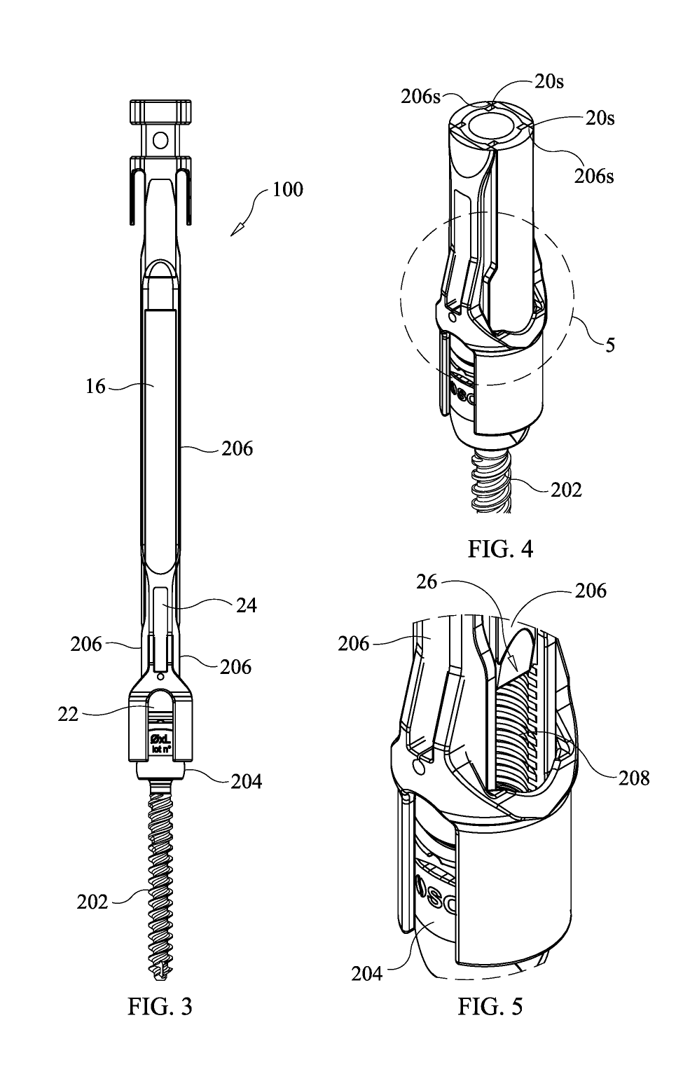

FIG. 1 is a perspective view of a sleeve 10 according to an embodiment of the present invention. Sleeve 10 has a distal end portion 10D that is configured and dimensioned to attach to the tulip portion of a bone fastener for manipulation thereof in performing procedures such as, but not limited to, reduction, compression, distraction and final tightening. The length 12 of sleeve 10 may vary depending upon multiple factors, including, but not limited to: location of the vertebrae that are being operated on; size of the patient; level of obesity of the patient, etc. Typical lengths 12 of sleeve 10 are within a range of from about 100 mm to about 220 mm, typically from about 140 mm to about 185 mm. The embodiment shown in FIG. 1 has a length of about 165 mm. Sleeve 10 is preferably made of stainless steel, but may alternatively be made from other rigid metals or polymers, including, but not limited to: titanium, polyether ether ketone (PEEK), carbon-fiber-reinforced polyether ether ketone (CFR-PEEK), cobalt chromium alloys, etc.

Sleeve 10 includes at least one channel 14 (the embodiment of FIG. 1 has one channel 14) that extends the length thereof, to allow tools to access a distal end portion of the sleeve 10 from a location external of the proximal end of the sleeve 10. The diameter of channel 14 typically is a value in a range of from about 5 mm to about 15 mm. In the embodiment shown in FIG. 1, the diameter of the channel 14 is about 7 mm. Sleeve 10 includes wall 16 that extends from the distal end portion 10D to a proximal end portion 10P that includes a tool interface 18. Slots 20 are formed in the wall 16 and are configured to receive extended tabs of the tulip of the bone fastener when sleeve 10 attaches to the bone fastener.

A passage 22 is formed through opposite walls of the distal end portion that is configured to allow a rod or other connector to pass therethrough so that it can be connected to adjacent bone fasteners.

Retainer 24 is provided adjacent distal end portion 10D and is configured to releasably retain a fixation member at the proper height and orientation to mate with a retention feature on the tulip of the bone fastener.

FIG. 2 shows an assembly 100 in which sleeve 10 has been attached to bone fastener 200 in an operational state. Bone fastener 200 as shown includes a threaded shaft 202 that is used to thread into the bone being treated to form an attachment thereto; a tulip 204 and extended tabs 206 extending from tulip 204. The slots 20 of sleeve 10 that are dimensioned to receive extended tabs form stops 20S that mate with the sides 206S of the extended tabs 206 to prevent rotation of the sleeve 10 relative to the extended tabs 206 and thus to also prevent rotation of the sleeve 10 relative to tulip 204, as illustrated in the cross-sectional view in FIG. 4 which is taken along line 4-4 in FIG. 2.

FIG. 5 is an enlarged, partially see-through, partial view of the assembly 100 taken from box 5 in FIG. 4. FIG. 5 shows an alignment feature 26 that is provided on the inner surface of wall 16 that is configured to properly align the orientation of sleeve 10 in the height direction when attached to the bone fastener 200. Alignment feature 26 can be a ledge, shoulder or the like, that contacts the top of the retention feature 208 and thereby prevents the sleeve 10 from sliding down any further along the tulip 204. Upon contact with the top of the tulip 204 by alignment feature 26, sleeve 10 is properly vertically aligned to line up the fixation member with the retention feature of the tulip so that mating between the fixation member and retention feature can be readily started and easily performed. In the embodiment shown in FIG. 5, retention feature 208 comprises threads that are configured to mate with mating threads on the fixation member. However, alternative types of mating features could be substitute, as would be readily apparent to those of ordinary skill in the art. It is noted that the retention feature 208, such as threads may optionally extend from the tulip 204 over a predetermined distance of the extended tabs 206.

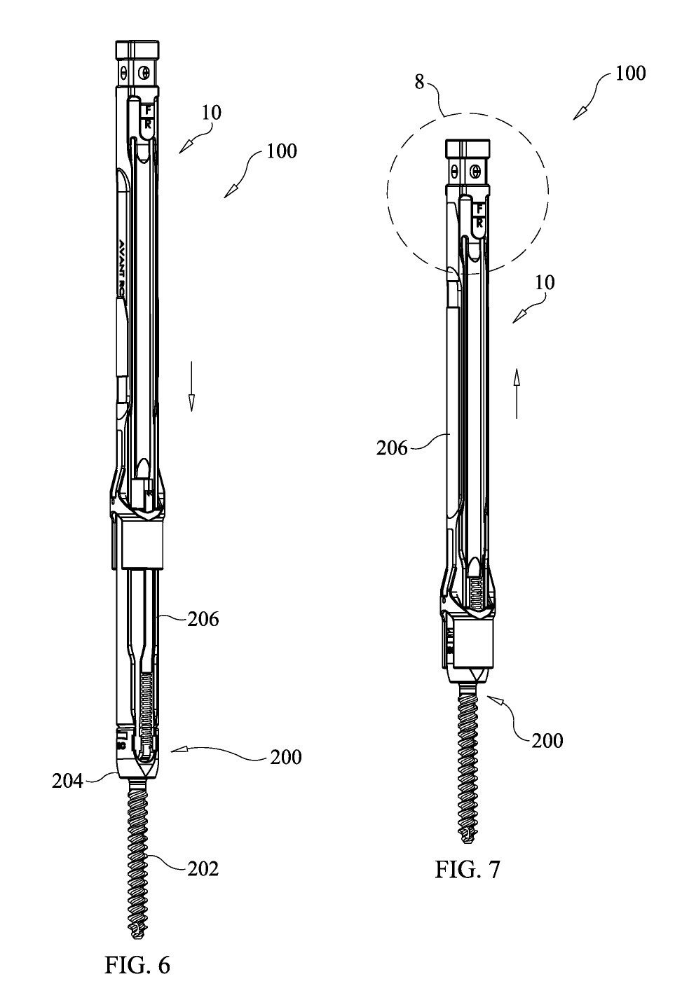

FIG. 6 illustrates an assembly process of 100 in progress. Although not shown, in typical use, the threaded shaft 202 will have already been threaded into bone to attach the bone fastener 200 to the bone prior to assembly of 100. To assemble, the slots 20 of sleeve 10 are aligned with the extended tabs 206 and then the sleeve 10 is pushed down over the bone fastener. As the sleeve 10 is pushed down, the extended tabs 206 are received in slots 20 and this process is continued until the fixation member 32 abuts the top of the retention feature 208, to form the assembly 100 illustrated in FIG. 7. Optionally, the distal end portion 10D may form a compression fit with the tulip 204.

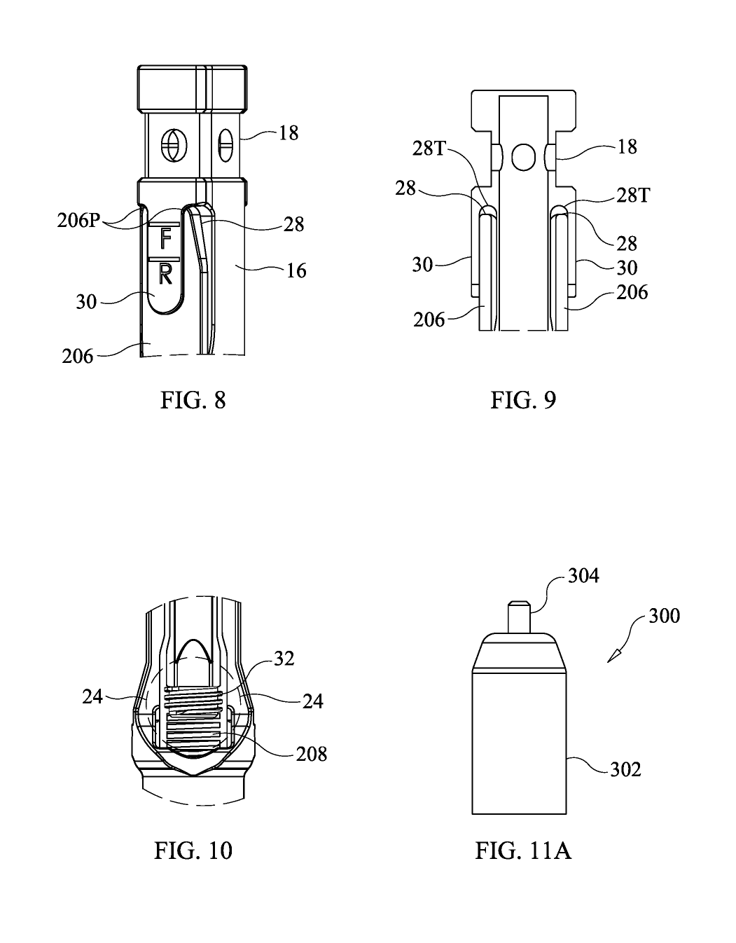

Upon completion of assembly, the proximal end portions of extended tabs 206 are received in slots 28 formed between stays 30 and the wall 16 of the sleeve 10. A detailed view of these features is shown in FIG. 8, which is an enlarged, partial view of the portion of FIG. 7 contained within box 8. It is noted that the proximal ends 206P may not make contact with the tops of the slots 28, or, alternatively, the slots 28 can be designed so that the proximal ends 206 make contact with the top ends 28T of the slots 28 to function as an additional (or alternative) vertical alignment feature. In either case, the capture of the proximal end portions 206P of the extended tabs 206 in the slots 28 and provision of stays (rigid tabs) 30 prevents splay or widening of the distance between the extended tabs during operations performed using the sleeve 10.

FIG. 9 shows a longitudinal sectional view of the portion shown in FIG. 8. As can be seen, in this embodiment, the proximal ends of extended tabs 206 do not contact the top ends 28T of slots 28.

Removal/disconnection of the sleeve 10 from the bone fastener 200 can be achieved by simply pulling on the sleeve 10 in the direction shown in FIG. 7 to slide the sleeve off of the tulip 204 and extended tabs 206.

Retainer 24, as noted above, is provided adjacent distal end portion 10D and is configured to releasably retain a fixation member at the proper height and orientation to mate with a retention feature on the tulip 204 (or on extended tabs 206 and extending into tulip 24) of the bone fastener 200. FIG. 10 is a detailed, partially transparent view of the portion of assembly 100 contained within circle 10-10 in FIG. 2. Fixation member 32 (which is a set screw in the embodiment shown in FIG. 10, but could be an alternative type of fixation member, as described above) is releasably retained by retainer 24 at a height relative to retention feature 208 to automatically align it with the retention feature 208 when sleeve 10 is installed on bone fastener 200 as described, so that fixation member is aligned exactly for ease of mating the fixation member 32 to the retention feature 208. In an embodiment where retention feature 208 and fixation member 32 are mating threads as shown in FIG. 10, the retainer 24 retains fixation member 32 at a height relative to the retention feature 208 so that the fixation member 32 is properly aligned and oriented such that rotation of the fixation member 32 (typically in a clockwise direction, but could be counter-clockwise) immediately engages the leading thread of the fixation member 32 with the first thread of the retention feature. In the embodiment shown, retainer 24 comprises a pair of leaf springs, but retainer 24 could alternatively be formed by other resilient members, such as coil springs, living hinges, ball and detent features, etc.

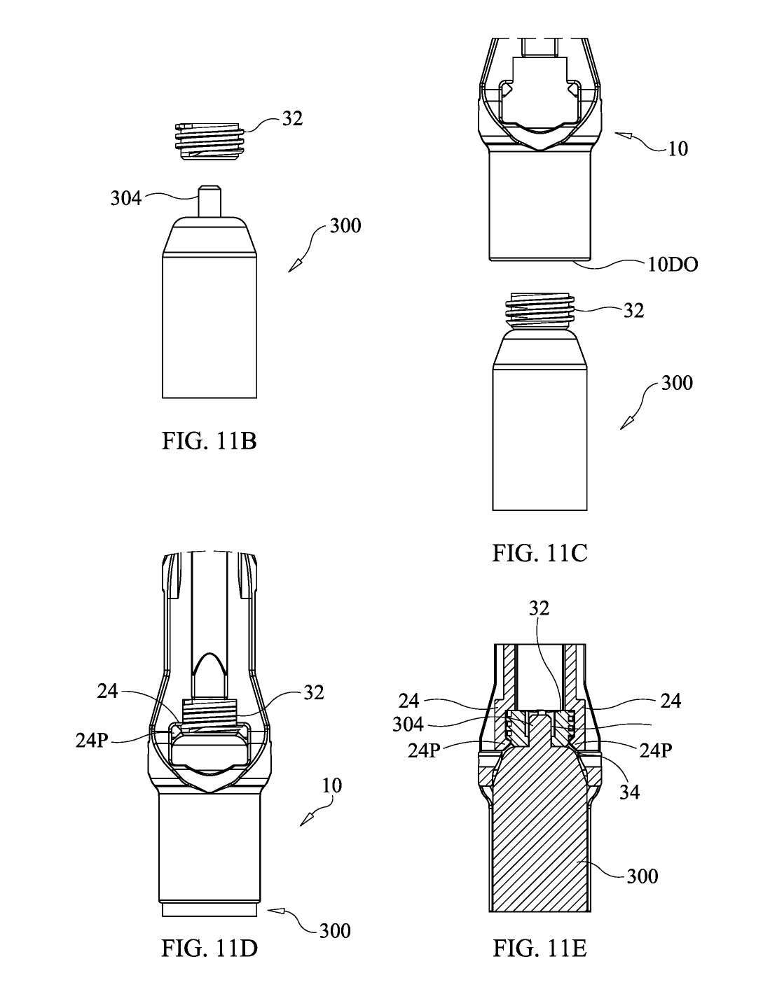

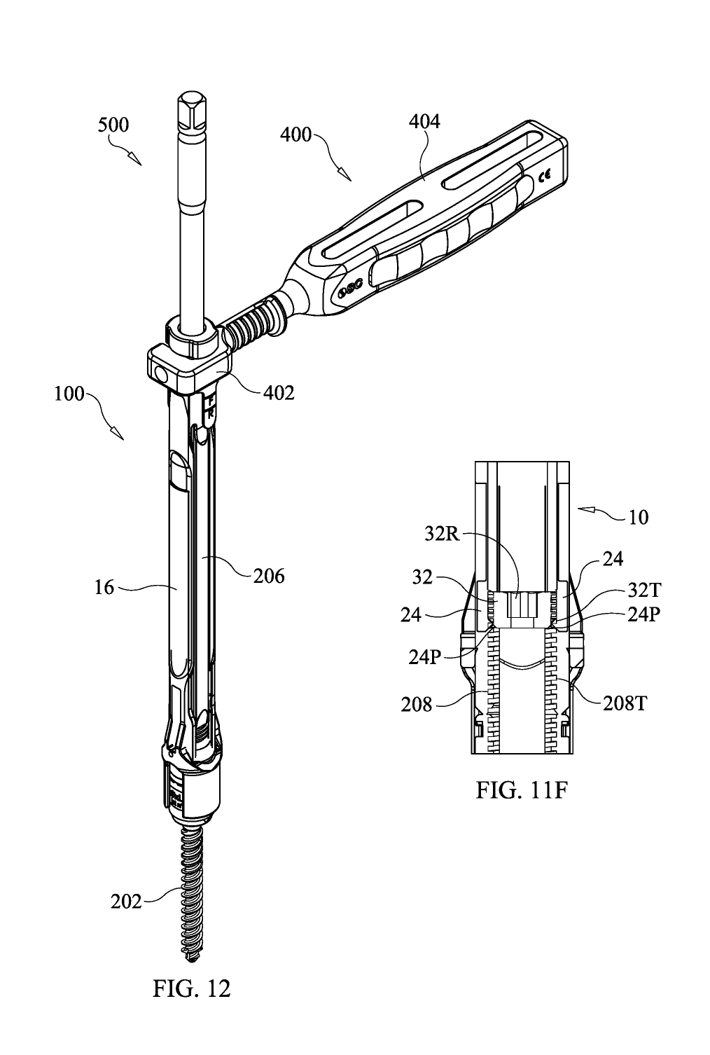

FIG. 11A shows a loading block 300 that is configured and dimensioned to load the fixation member 32 into the sleeve 10 for reliable retention thereof by the retainer 24. Loading block 300 comprises a main elongate body 302 that is dimensioned to be received through the distal opening of sleeve 10 and to be slidably received in the channel 14. In the embodiment shown, main body 302 is cylindrical, but alternatively, could have other cross-dimensional shapes, such as oval, elliptical, square, hexagonal, asymmetric, or any other shape as long as the dimensions thereof allow it to be received in the channel 14. A loading pin 304 extends proximally from the proximal end of main body 302 and is configured and dimensioned for slidably receiving the fixation member 32 as it is mounted thereon.

FIG. 11B illustrates fixation member 32 being mounted on the loading block 300. Loading pin 304 is dimensioned to pass through opening 34 (see FIG. 11E) of fixation member 32 so as to slidably receive the fixation member 32 thereover and form a close fit with the wall forming the opening 34. This close fit also maintains the longitudinal axis of the fixation member 32 in alignment with the longitudinal axis of the loading block 302, which will also ensure that the longitudinal axis of the fixation member 32 is aligned with the longitudinal axis of the retainer 24. This is important for ensuring that the threads of the fixation member 32 and of the retention feature are properly aligned to prevent cross-threading.

FIG. 11C shows fixation member 32 mounted on loading block 300 and loading block 300 aligned with sleeve 10 in preparation for loading the fixation member 32 into the sleeve 10. The fixation member 32 and loading block are passed through the distal opening 10DO of the sleeve. The loading block 300 is advanced into the sleeve 10 and when fixation member 32 abuts prongs 24P of retainer 24, the prongs 24 and retainer 24 are deflected outwardly as the fixation member 32 and loading block 300 continue their advancement.

Once the fixation member 32 has been properly positioned (both height wise and in alignment with the longitudinal axis of the sleeve 10), the retainer 24 resiliently returns to its unbiased position and prongs 24P retain the fixation member 32 at the desired relative height and orientation relative to the sleeve 10, as shown in FIG. 11E. The loading block 300 is then slid out of the sleeve 10, leaving the fixation member 32 in releasable retention by the retainer 24 at the desired location and orientation. The retainer 24 is configured in the sleeve 10 to releasably retain the fixation member 32 at predetermined distance from the distal end of the sleeve that is predetermined to place the distal end of the fixation member 32 in alignment with the proximal end of the retention feature 208, so that no manipulation of the fixation member 32 is needed (other than the driving motion for mating the fixation member 32 with the retention feature 208) after assembling the sleeve 10 on the bone fastener 200.

As shown in the longitudinal sectional view of FIG. 11F, threads 32F of fixation member 32 are aligned and oriented with threads 208T of retention feature 208 so that the leading thread of threads 32T is positioned to mate with the leading thread of threads 208T immediately upon rotating fixation member 32 relative to the sleeve 10 and retention feature 208. Upon mating with the threads 208T, continued rotation of fixation member 32 draws the fixation member 32 past the prongs 24P, forcing prongs 24P and retainer to resiliently open, thereby releasing the fixation member 32 as it is received in the retention feature. Upon full mating of the fixation member 32 with the retention feature 208, the fixation member 32 clears the prongs 24P and the prongs 24P and retainer 24 resiliently return to their unbiased positions.

As known conventionally, the fixation member 32 can then be used to be torqued own on a rod or the like for fixation thereof (final fixation). Prior to such final fixation, sleeve 10 can be used to facilitate the performance of manipulation of the bone fastener 200, such as in the performance of reduction, distraction and/or compression, or other manipulations.

In one embodiment of use in a minimally invasive surgical procedure, a patient is prepared for a spinal procedure. A skin incision (e.g., stab wound) is made through the back of the patient and a needle is inserted through fascia to contact a pedicle of a vertebra. The inner stylet of needle is removed and a guidewire is insert through the needle. A bone awl may optionally be used to penetrate cortical bone of pedicle. A tap may be used to tap threads into the pedicle.

Progressively larger dilators can be inserted over the guidewire to dilate the opening. The dilator(s) is removed and the bone fastener 200 is fastened to the pedicle using an minimally-invasive torquing tool. The tool is removed and the guidewire is removed. A rod may be installed through passage 22 either before or after assembling sleeve 10 on the bone fastener 200.

Prior to assembling the sleeve 10 on the bone fastener 200, a fixation member 32 is loaded into sleeve 10 to be releasably retained by retainer 24, as described above. Once the fixation member 32 has been releasably retained by retainer 24 at the proper orientation and height, sleeve 10 is next installed over the extended tabs 206 and tulip 204 of the bone fastener 200 in a manner as described above. This positions fixation member 32 at the proper height and orientation to readily engage and mate with retention feature 208.

Once assembled, the assembly 100 can be used to perform reduction, distraction and/or compression, as well as other manipulations of the bone fastener as needed utilizing the sleeve 10. The fixation member 32 and retention feature 208 can be used to facilitate reduction.

A counter torque wrench 400 can be used to prevent rotation of the sleeve 10/bone fastener 200 while the fixation member 32 is being torqued into the retention feature 208. The fixation member 32 can be torqued by inserting a driving tool 500 through the channel 14 to engage with a tool receiving feature 32R in the proximal end of the fixation member 32 that is configured to mate with a working end of the tool 500, and rotating the tool 500 relative to the sleeve 10. Counter-torque wrench 400 includes a working end 402 configured to mate with tool interface 18 of sleeve 10, and an elongate handle 404 designed to be grasped by the user and to provide a mechanical advantage (leverage) to the interface between working end 402 and tool interface 18.

Once all manipulations of the bone fastener 204 using sleeve 10 have been performed satisfactorily, a final tightening (final fixation) of the fixation member 32 against the rod is performed, while providing counter-torque with tool 400.

The sleeve 10 can then be removed from the patient and the bone fastener 200. The fixation member 32 having mated with the portion of the retention feature in tulip 204, extended tabs 206 can then be broken off and removed from the patient.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A sleeve for performing surgical procedures, said sleeve comprising:
an elongate main body;
a channel extending within a length of said elongate main body; and
a retainer configured and dimensioned to releasably retain a fixation member in said sleeve at a predetermined orientation and predetermined distance from a distal end of said sleeve.

2. The sleeve of claim 1, wherein said retainer is resiliently deformable to receive the fixation member.

3. The sleeve of claim 2, wherein said retainer comprises at least one leaf spring.

4. The sleeve of claim 3, wherein said retainer comprises a pair of opposing leaf springs.

5. The sleeve of claim 2, wherein said retainer comprises at least one prong to releasably capture said fixation member.

6. The sleeve of claim 1, wherein a distal end portion of said sleeve is configured and dimensioned to attach to a tulip portion of a bone fastener.

7. The sleeve of claim 1, wherein said sleeve further comprises a tool interface configured to mate with a working end of a counter-torque tool.

8. The sleeve of claim 1, further comprising a passage formed through opposite walls of a distal end portion of said sleeve, said passage being configured to allow a rod or other connector to pass therethrough.

9. The sleeve of claim 1, further comprising slots in said elongate main body configured and dimensioned to receive extended tabs that extend from a bone fastener.

10. The sleeve of claim 9, wherein said slots comprise stops that are configured to mate with sides of the extended tabs to prevent rotation of said sleeve relative to the extended tabs.

11. The sleeve of claim 1, further comprising an alignment feature provided on an inner surface of a wall of said elongate main body, configured to properly align an orientation of said sleeve in a height direction relative to a bone fastener, when said sleeve is attached to the bone fastener.

12. The sleeve of claim 11, wherein said alignment feature comprises a ledge or shoulder.

13. The sleeve of claim 9, further comprising stays forming anti-splaying slots with the a wall of said elongate main body, wherein said anti-splaying slots are configured to receive proximal end portions of the extended tabs and said stays are configured to prevent splaying of the extended tabs.

14. The sleeve of claim 1, further comprising said fixation member releasably retained by said retainer at said predetermined orientation and predetermined distance from said distal end of said sleeve.

15. The sleeve of claim 14, wherein said fixation member comprises a set screw.

16. A sleeve for performing surgical procedures, said sleeve comprising:
an elongate main body;
a channel extending within a length of said elongate main body; and
slots in said elongate main body configured and dimensioned to receive extended tabs that extend from a bone fastener; and
an alignment feature provided on an inner surface of a wall of said elongate main body, configured to properly align an orientation of said sleeve in a height direction relative to a bone fastener, when said sleeve is attached to the bone fastener.

17. The sleeve of claim 16, wherein said slots comprise stops that are configured to mate with sides of the extended tabs to prevent rotation of said sleeve relative to the extended tabs.

18. The sleeve of claim 16, wherein said alignment feature comprises a ledge or shoulder.

19. The sleeve of claim 16, further comprising stays forming anti-splaying slots with the a wall of said elongate main body, wherein said anti-splaying slots are configured to receive proximal end portions of the extended tabs and said stays are configured to prevent splaying of the extended tabs.

20. An assembly for performing surgical procedures, said assembly comprising:
a sleeve comprising:
an elongate main body; and
a channel extending within a length of said elongate main body;
a fixation member having been preloaded in said sleeve; and
a bone fastener comprising:
a shaft configured to engage bone;
a tulip configured to receive a rod;
a retention feature; and
extended tabs extending from said tulip;
wherein a distal end portion of said sleeve is mounted over said tulip;
wherein said extended tabs are received in slots in said sleeve; and
wherein said fixation member is located at a predetermined orientation and distance from a distal end of said sleeve to approximate a proximal end of said retention feature.

21. The assembly of claim 20, wherein said sleeve further comprises a retainer that releasably retains said fixation member in said sleeve at said predetermined orientation and said predetermined distance from said distal end of said sleeve.

22. The assembly of claim 21, wherein said retainer is resiliently deformable to receive the fixation member.

23. The assembly of claim 20, wherein said sleeve further comprises a tool interface configured to mate with a working end of a counter-torque tool.

24. The assembly of claim 23, further comprising a counter-torque tool, said working end of said counter-torque tool engaged with said tool interface.

25. The assembly of claim 20, wherein said sleeve further comprises a passage formed through opposite walls of said distal end portion of said sleeve, said passage being configured to allow the rod or other connector to pass therethrough.

26. The assembly of claim 20, further comprising slots in said elongate main body, wherein said extended tabs are received in said slots.

27. The assembly of claim 26, wherein said slots comprise stops that mate with sides of said extended tabs to prevent rotation of said sleeve relative to said extended tabs.

28. The assembly of claim 20, wherein said sleeve further comprises an alignment feature provided on an inner surface of a wall of said elongate main body, configured to properly align an orientation of said sleeve in a height direction relative to said bone fastener.

29. The assembly of claim 20 wherein said sleeve further comprises stays forming anti-splaying slots with the a wall of said elongate main body, wherein said anti-splaying slots receive proximal end portions of said extended tabs and said stays prevent splaying of said extended tabs.

30. The assembly of claim 20, wherein said fixation member comprises a set screw.

31. A method of performing a surgical procedure, said method comprising:
providing an elongate sleeve having proximal and distal ends and a channel extending therethrough;
releasably retaining a fixation member in said channel of said sleeve at a predetermined orientation and predetermined distance from said distal end of said sleeve; and
assembling said sleeve on a bone fastener.

32. The method of claim 31, wherein said fixation member is preloaded into said channel prior to said assembling.

33. The method of claim 30, wherein said fixation member is resiliently and releasably retained by a retainer.

34. The method of claim 33, wherein said retainer is spring loaded.

35. The method of claim 30, wherein said fixation member comprises a set screw.

36. The method of claim 32, wherein said fixation member is preloaded by:
mounting said fixation member on a loading block; and
advancing said fixation member and said loading block into said channel from said distal end of said sleeve.

37. The method of claim 36, wherein said fixation member is advanced against a retainer that resiliently deforms to allow said fixation member to pass by and resiliently returns to an unbiased configuration to releasably retain said fixation member.

38. The method of claim 31, further comprising:
mating said fixation member with a retention member of said bone fastener.

39. The method of claim 38, further comprising performing an operation on said bone fastener by manipulating said sleeve.

40. The method of claim 39, wherein said operation comprises at least one of reduction, distraction or compression.

41. The method of claim 39, further comprising finally fixing said fixation member in said tulip.

42. The method of claim 41, further comprising removing said sleeve from said bone fastener and breaking off said extended tabs.
